(19) United States
(12) Patent Application Publication
(10) Pub. No.: US 2022/0170946 A1
(43) Pub. Date: Jun. 2, 2022

(54) METHOD OF DETECTING, DETERMINING, AND TREATING HEAT STRESS BASED ON EXPRESSION OF GRP75 IN AVIAN SPECIES

(71) Applicant: Board of Trustees of the University of Arkansas, Little Rock, AR (US)

(72) Inventor: Sami Dridi, Fayetteville, AR (US)

(73) Assignee: Board of Trustees of the University of Arkansas, Little Rock, AR (US)

(21) Appl. No.: 17/506,743

(22) Filed: Oct. 21, 2021

Related U.S. Application Data
(60) Provisional application No. 63/094,635, filed on Oct. 21, 2020.

(51) Int. Cl. G01N 33/68; C12Q 1/6883; A23K 50/75

(52) U.S. Cl. CPC G01N 33/6893; G01N 2800/40; A23K 50/75; C12Q 1/6883

(57) ABSTRACT

This invention relates generally to a method of detecting, determining, and treating heat stress in avian species based on expression of GRP75. More particularly, the invention relates to a non-invasive and accurate process to evaluate heat stress in avian and poultry species, such as broiler chickens, layers, turkeys, breeders, and/or quail. The method uses GRP75 expression as a non-invasive marker for heat stress in avian species, and also provides for a method to rapidly evaluate and continuously treat and monitor dynamic change and time-course stress induced by heat stress load in the same individual poultry.

### CROSS REFERENCE TO RELATED APPLICATIONS

This application claims the benefit of U.S. Provisional Patent Application Ser. No. 63/094,635 filed Oct. 21, 2020, and incorporates said provisional application by reference into this document as if fully set out at this point.

### STATEMENT REGARDING PRIOR DISCLOSURES BY THE INVENTOR

This application incorporates by reference Dhamad, et al. 75-kDa Glucose-Regulated Protein (GRP75) Is a Novel Molecular Signature for Heat Stress Response in Avian Species; Am J Physiol Cell Physiol, 318(2):C289-C303, Dec. 4, 2020, into this document as if fully set out at this point.

### SEQUENCE LISTING

This application includes an electronically submitted sequence listing in .txt format. The .txt file contains a sequence listing entitled “GRP75 ST25.txt” created on Oct. 14, 2021, and is six (6) kilobytes in size. The sequence listing contained in this .txt file is part of the Specification and is hereby incorporated by reference herein in its entirety.

### BACKGROUND OF THE INVENTION

### 1. Field of the Invention

This invention relates generally to a method of detecting, determining, and treating stress induced by heat load in avian species based on expression of GRP75, and more particular to a method of using GRP75 gene and protein expression as a non-invasive marker for heat stress in avian and poultry species.

### 2. Description of the Related Art

Animal agriculture and particularly poultry is facing substantial challenges from a steep projected increase in demand for high quality animal protein and the need to adapt to higher temperatures due to climate change. Large, abrupt, and widespread extreme heat waves have occurred repeatedly in the past and are predicted to increase for the next century.

Environmental heat stress impacts every aspect of animal life and their very existence. The effects of environmental heat stress are particularly devastating to modern poultry production. Heat stress adversely affects avian feed intake, growth performance, welfare, leaky gut syndrome, immunosuppression, meat yield and quality, and mortality. Such effects will take a heavy toll during the next decades as the distribution of heat anomalies continues to increase.

The U.S. poultry industries lose over $350 million annually, and global livestock loses are in the billions. For instance, in 2011, a heat wave swept across the eastern United States, killing hundreds of thousands of chickens and turkeys. The European heat wave of summer 2003 resulted in over one million chicken deaths in France. In the 2015 summer months, a month-long heat wave in India escalated the poultry mortality rate from an average of 2-3% to almost 10%, or 17 million birds. According to a 2003 analysis, the American animal agriculture industry loses an estimated $1.69 to $2.36 billion annually due to heat shock, with poultry-specific losses ranging from $128 to $165 million. Additionally, as global temperatures (intensity and duration) are predicted to increase over the coming decades, these negative events are projected to have an even greater impact on animal health and performance, economic losses, and food security for a growing world population.

The animal agriculture industry currently attempts to mitigate the negative impacts of heat stress through managerial (cooling systems) and nutritional (vitamins E, A, etc.) strategies. However, avian mortality rates and economic losses are still significant during hot weather. Failure to effectively combat heat stress is mainly due to a lack of fundamental and basic knowledge of heat stress responses at molecular and cellular levels.

It is therefore desirable to identify molecular markers that can be used to detect heat stress in avian species. In particular, a need exists for reliable markers to help identify and combat heat stress in poultry species, particularly with modern broilers, to reduce losses to the animal agriculture industry.

It is further desirable to provide a method of non-invasively detecting and determining heat stress in poultry based on expression of GRP75.

It is still further desirable to provide a method of using GRP75 gene and protein expression as a non-invasive marker for heat stress in avian and poultry species.

It is yet further desirable to provide a method to rapidly evaluate, mitigate, and continuously monitor dynamic change and time-course stress induced by heat load in the same individual poultry.

Before proceeding to a detailed description of the invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

In general, the invention relates to GRP75 protein as a novel molecular marker for detecting heat stress in avian species. The chicken GRP75 protein has been identified by using an immunoprecipitation assay integrated with LC-MS/MS technique, and immunofluorescence staining has shown that the GRP75 protein is predominantly localized in avian mitochondria. Chicken GRP75 has an amino acid sequence conserved with high homology (52.5%) to the heat-shock protein 70 family (HSP70). Bioinformatics and 3D-structure prediction indicate that, like most HSPs, chicken GRP75 has two principal domains: the N-terminal ATPase and C-terminal region. Chicken GRP75, unlike its mammalian orthologs, is responsive to heat shock and plays a key role in cell death and survival pathways during heat stress. Heat stress exposure upregulates chicken GRP75, causing a reduction in avian cell viability as GRP75 protein becomes overexpressed. Blockade of GRP75 by its small molecular inhibitor, MKT-077, rescues avian cell viability during heat stress. Therefore, GRP75 protein provides a novel molecular tool for determining heat stress response in avian species, such as chickens, turkeys, and quail.

More particularly, the invention relates to non-invasive methods of detecting, determining, and treating stress in avian species, namely poultry. The invention further relates to a polymerase chain reaction (PCR) GRP75 kit or an enzyme-linked immunosorbent assay (ELISA) GRP75 kit for use with the method of detecting and determining stress in avian species disclosed herein.

The foregoing has outlined in broad terms some of the more important features of the invention disclosed herein so that the detailed description that follows may be more clearly understood, and so that the contribution of the inventors to the art may be better appreciated. The invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1A is a Western blot performed using a monoclonal anti-GRP75 antibody, which resulted in only one band of GRP75 at the expected molecular weight (75 kDa). Recombinant human GRP75 (rhGRP75) was used as a positive control.

FIG. 1B is a predicted 3D structure of chicken GRP75 showing ATP- and peptide-binding domains. This 3D structure was generated through a PyMOL homology modeling program. The 675 amino acids used for prediction were determined by liquid chromatography coupled with tandem mass spectrometry (LC-MS/MS) analysis and directly submitted to PRIDE and ProteomeXchange Knowledgebase (PRIDE, EMBL-EBI, Accession number # PXD013591, DOI 10.6019/PXD013591).

FIG. 1C is a predicted 3D structure demonstrating that the chicken GRP75 structure matches that of heat shock protein family A (HSPAS) with high homology (52.5%), indicating relation to the HSP70 family.

FIG. 2A depicts double immunofluorescence staining of DAPI for DNA.

FIG. 2B depicts double immunofluorescence staining of the avian cell line spontaneously immortalized chicken embryonic hepatocyte (siCEH).

FIG. 2C depicts double immunofluorescence staining of mitotracker for mitochondria.

FIG. 2D depicts double immunofluorescence staining of siCEH with DAPI as a stain for DNA and mitotracker as a stain for mitochondria. Arrows indicate colocalization.

FIG. 2E depicts double immunofluorescence staining of DAPI for DNA.

FIG. 2F depicts double immunofluorescence staining of the avian cell line quail myoblast QM7.

FIG. 2G depicts double immunofluorescence staining of mitotracker for mitochondria.

FIG. 2H depicts double immunofluorescence staining of QM7 with DAPI as a stain for DNA and mitotracker as a stain for mitochondria. Arrows indicate colocalization.

FIG. 3A is a Western blot, representative of three replicates, that depicts chicken GRP75 protein expression in liver following heat stress. HS, heat stress; TN, thermoneutral.

FIG. 3B graphically illustrates upregulation of chicken GRP75 protein expression in liver following heat stress. Data are mean ±SEM. * indicates a significant difference compared to thermoneutral birds.

FIG. 3C is a Western blot, representative of three replicates, that depicts chicken GRP75 protein expression in muscle following heat stress. HS, heat stress; TN, thermoneutral.

FIG. 3D graphically illustrates upregulation of chicken GRP75 protein expression in muscle following heat stress. Data are mean ±SEM. * indicates a significant difference compared to thermoneutral birds.

FIG. 4A graphically illustrates that overexpression of GRP75 reduces viability of siCEH cells. HS, heat stress; siCEH, spontaneously immortalized chicken embryonic hepatocyte. Cell viability was determined by flow cytometry. Data are mean ±SEM. * * * indicates a significant difference compared to pNull at P<0.001.

FIG. 4B graphically illustrates blockade of GRP75 by small molecular inhibitor MKT-077. MKT-077, 1-ethyl-2-[[3-ethyl-5-(3-methyl-2(3H)-benzothiazolylidene)-4-oxo-2-thiazolidinylidene]-methyl]-pyridinium chloride. Data are mean ±SEM. * indicates a significant difference compared to MKT-077 at P<0.05.

FIG. 4C graphically illustrates that blockade of GRP75 by small molecular inhibitor MKT-077 rescues cell viability under 2-h heat stress exposure. HS, heat stress; siCEH, spontaneously immortalized chicken embryonic hepatocyte; MKT-077, 1-ethyl-2-[[3-ethyl-5-(3-methyl-2(3H)-benzothiazolylidene)-4-oxo-2-thiazolidinylidene]-methyl]-pyridinium chloride. Data are mean ±SEM. Different superscript letters indicate a significant difference at P<0.05.

FIG. 4D graphically illustrates that blockade of GRP75 by small molecular inhibitor MKT-077 rescues cell viability under 2-h heat stress exposure. Cell viability was measured using 7-aminoactinomycin D (7-AAD) staining and FACScan flow cytometry. Dead-cell populations penetrated by 7-AAD were measured on the FL3 fluorescent channel using a minimum of 10,000 events, and data analysis was performed by FlowJO software.

FIG. 5A depicts double immunofluorescence staining of the avian cell line spontaneously immortalized chicken embryonic hepatocyte (siCEH) showing the localization of GRP75 in the endoplasmic reticulum co-stained with DAPI and ERGIC53. Arrows indicate colocalization.

FIG. 5B depicts double immunofluorescence staining of siCEH showing the localization of GRP75 in the Golgi complex co-stained with DAPI and TGN38. Arrows indicate colocalization.

FIG. 5C depicts double immunofluorescence staining of siCEH showing the localization of GRP75 in the endoplasmic reticulum co-stained with DAPI and ER-tracker. Arrows indicate colocalization.

FIG. 5D depicts double immunofluorescence staining of siCEH showing the localization of GRP75 in the Golgi complex co-stained with DAPI and BiP. Arrows indicate colocalization.

FIG. 5E depicts double immunofluorescence staining of the avian cell line myoblast QM7 showing the localization of GRP75 in the endoplasmic reticulum co-stained with DAPI and ERGIC53. Arrows indicate colocalization.

FIG. 5F depicts double immunofluorescence staining of QM7 showing the localization of GRP75 in the Golgi complex co-stained with DAPI and TGN38. Arrows indicate colocalization.

FIG. 5G depicts double immunofluorescence staining of QM7 showing the localization of GRP75 in the endoplasmic reticulum co-stained with DAPI and ER-tracker. Arrows indicate colocalization.

FIG. 5H depicts double immunofluorescence staining of QM7 showing the localization of GRP75 in the Golgi complex co-stained with DAPI and BiP. Arrows indicate colocalization.

### DETAILED DESCRIPTION OF THE INVENTION

While this invention is susceptible of embodiment in many different forms, there is shown in the drawings, and will herein be described hereinafter in detail, some specific embodiments of the invention. It should be understood, however, that the present disclosure is to be considered an exemplification of the principles of the invention and is not intended to limit the invention to the specific embodiments so described.

This invention relates generally to a method of detecting, determining, and treating stress in avian species based on expression of GRP75. More particularly, the invention relates to a non-invasive and accurate process to evaluate heat stress in avian and poultry species, such as broiler chickens, layers, turkeys, breeders, and/or quail. The method uses GRP75 expression as a non-invasive marker for heat stress in avian species, and also provides for a method to rapidly evaluate, implement heat mitigation strategies, and continuously monitor dynamic change and time-course stress induced by stress load in the same individual poultry. The method can measure the time-course and dynamic change of stress levels in the same individual without biopsy or animal euthanasia.

The inventive method includes non-invasively obtaining a sample from a living avian species or subject without biopsy or euthanasia. A polymerase chain reaction kit, an enzyme-linked immunosorbent assay (ELISA) kit or the like is then used to detect the presence of GRP75 in the sample. The sample is assayed for the expression level of a heat stress-related marker, with the marker being a protein encoded by the gene of GRP75 or fragments of the protein which are immunoreacitve to GRP75. In addition, the amount of GRP75 expressed in the sample can be determined, and an elevated amount of GRP75 indicates the presence of heat stress in the avian species. Lastly, the method may include diagnosing the avian species with stress when GRP75 is detected in the sample isolated from the sample.

In addition, the invention can include an ELISA or a PCR GRP75 kit configured for detecting and determining heat stress in poultry based on expression of GRP75.

The chicken GRP75 protein is characterized by its key function in the death and survival of cells in avian species following exposure to heat stress. GRP75 is predominantly localized within mitochondria of hepatic and muscle avian cells. Mitochondria are a primary site for ATP synthesis, reactive oxygen species production during heat stress exposure, and oxidative stress. GRP75 plays an essential role within the import machinery and protein quality control of the mitochondria by regulating mitochondrial stress response signaling and by maintaining mitochondrial homeodynamics. Depending upon cell type, context, and/or stress type, severity, and duration, GRP75 has been shown to function as a guardian, a killer, or as a housekeeper within cells.

The GRP75 protein-coding gene, HSPA9B, is located on chromosome 13 of the chicken genome sequence assembly (Gallus_gallus-5.0). The chicken GRP75 protein consists of 675 amino acids. This amino acid sequence contains ATP- and peptide-binding domains that cover the positions from amino acid residues 51 to 434 and residues 435 to 581, respectively. The predicted binding structure of GRP75 illustrates that the protein binds ATP molecules through a designated ATP-binding pocket. Besides having two principal binding domains (the NH₂-terminal ATPase and COOH-terminal region), like most HSPs, the chicken GRP75 structure matches that of heat shock protein family A (HSPAS) with high homology (52.5%).

The validated chicken GRP75 amino acid sequence is identical to that of the NCBI reference sequence of Gallus gallus domesticus: NM_0010006147.1. The chicken GRP75 amino acid sequence is highly conserved, with >80% homology with the following species, for example: swine (Sus scrofa, accession no. XP_005661752), mouse (Mus musculus, accession no. NP_034611), rat (Rattus norvegicus, accession no. NP 001094128), human (Homo sapiens, accession no. NP_004125), bovine (Bos taurus, accession no. NP_001029696), lizard (green anole, Anolis carolinensis, accession no. ENSACAG00000015869), western clawed frog (Xenopus tropicalis, accession no. XB_GENE-960228), and gilt-head sea bream (Sparus aurota, accession no. A9CD13-1). [Note: These amino acid sequences were aligned and compared using the Clustal Omega program (EMBL-EBI)].]

However, unlike mammalian GRP75, chicken GRP75 protein is highly responsive to heat stress. This unique response to stress makes chicken GRP75 protein a novel molecular marker for heat and oxidative stress in avian species.

Acute heat stress induces expression of GRP75 protein within both liver and muscle in chicken and quail. The liver and muscle play key roles in metabolism, as well as with antioxidant systems, detoxification of drugs and xenobiotics, and thermogenesis. GRP75 expression is also induced in the hypothalamus of quail in response to heat stress, though it seems that this hypothalamic expression is species dependent.

Depending on a variety of factors—including duration, exposure, and severity—the effects of heat stress on poultry can range from discomfort (welfare) to organ damage to death by spiraling hyperthermia. In both siCEH (chicken) and QM7 (quail) cells, overexpression of GRP75 alone reduces cell viability. Conversely, blockade of GRP75 by its small molecular inhibitor, MKT-007, rescues cell viability of both siCEH and QM7 cells during heat stress. Therefore, levels of GRP75 protein relate directly to avian cell death and survival in response to heat stress.

Due to the correlation of GRP75 levels to cell viability after heat stress exposure, the GRP75 protein offers an inventive molecular marker for treating the impacts of heat stress on avian and poultry species, such as broiler chickens, layers, turkeys, breeders, and/or quail.

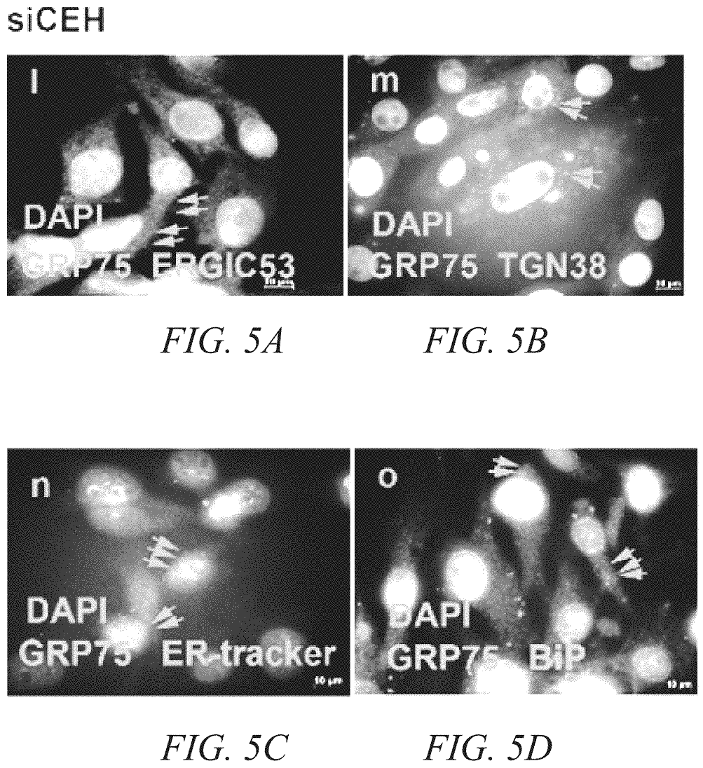

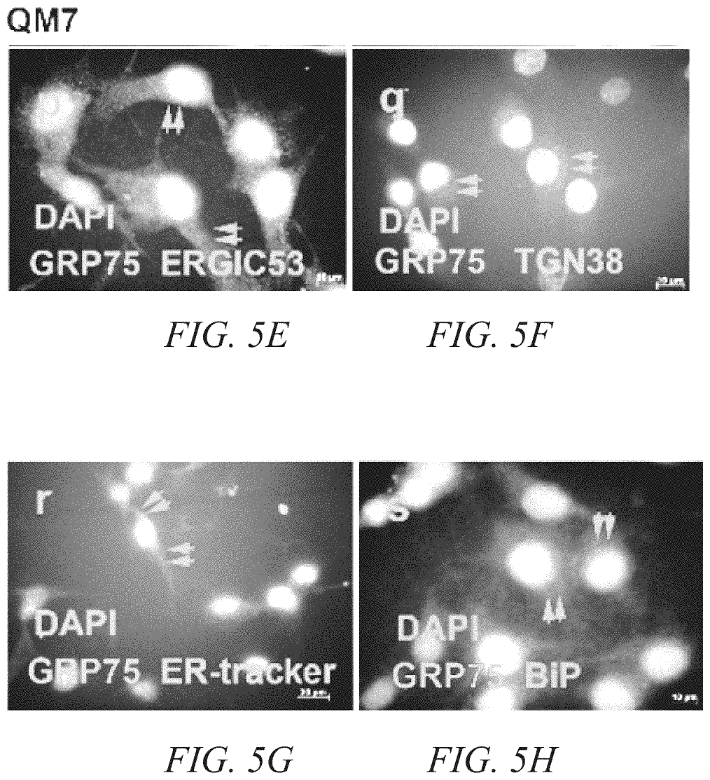

The GRP75 protein is also localized in the endoplasmic reticulum and the Golgi apparatus (FIGS. 5A-5H), and as such, maybe secreted in the circulation of avian species. The GRP75 protein marker can be incorporated into an ELISA kit to measure plasma GRP75 for practical monitoring. The ELISA kit could also be used to measure feather GRP75.

This non-invasive method based on the presence of GRP75 in blood, feathers, or otherwise would permit monitoring of heat stress response and well-being of the same bird over time during heat load. The method can measure time-course and dynamic changes of stress levels in the same individual without biopsy or animal euthanasia. In addition, after measuring feather GRP75, the bird's heat stress could be treated through managerial strategies, such as house design and location (e.g., naturally ventilated open-type housing, closed housed systems equipped with air conditioning, cooling pads, cool perches, and exhaust fans), restricted feeding, intermittent feeding, feeding during cool hours of the day, controlled environmental conditions, cooling systems (e.g., fans (either suspended from the interior building structures or vertical ceiling fans), interior fogging, and sprinkling systems), transporting, litter management, ventilation, and providing cold water. The bird's heat stress could also be treated through nutritional strategies, such as restricting the feed, wet or dual feeding, adding fat in diets, increasing energy in rations, supplementing vitamins, minerals, and electrolytes (e.g., vitamin E, A, C, zinc, chromium, selenium, sodium bicarbonate, KCL, lycopene), osmolytes (e.g., betaine or taurine), and phytochemicals (e.g., lycopene, resveratrol, epigallocatechin gallate (EGCG), curcumin). Moreover, the GRP75 could also be used as a genetic selection marker for thermo-tolerance in avian and other species.

### EXAMPLES

The methods of detecting and monitoring stress induced by heat load in avian species based on expression of GRP75 disclosed herein is further illustrated by the following examples, which are provided for the purpose of demonstration rather than limitation.

The chicken GRP75 protein has been identified by using an immunoprecipitation assay integrated with LC-MS/MS technique, and immunofluorescence staining has shown that the GRP75 protein is predominantly localized in avian mitochondria. Chicken GRP75 has an amino acid sequence conserved with high homology (52.5%) to the heat-shock protein 70 family (HSP70). Bioinformatics and 3D-structure prediction indicate that, like most HSPs, chicken GRP75 has two principal domains: the N-terminal ATPase and C-terminal region. Chicken GRP75, unlike its mammalian orthologs, is responsive to heat shock and plays a key role in cell death and survival pathways during heat stress. Heat stress exposure upregulates chicken GRP75, causing a reduction in avian cell viability as GRP75 protein becomes overexpressed. Blockade of GRP75 by its small molecular inhibitor, MKT-077, rescues avian cell viability during heat stress. Therefore, GRP75 protein provides a novel molecular tool for determining heat stress response in avian species, such as chickens, turkeys, and quail.

Characterization of Chicken GRP75 Protein.

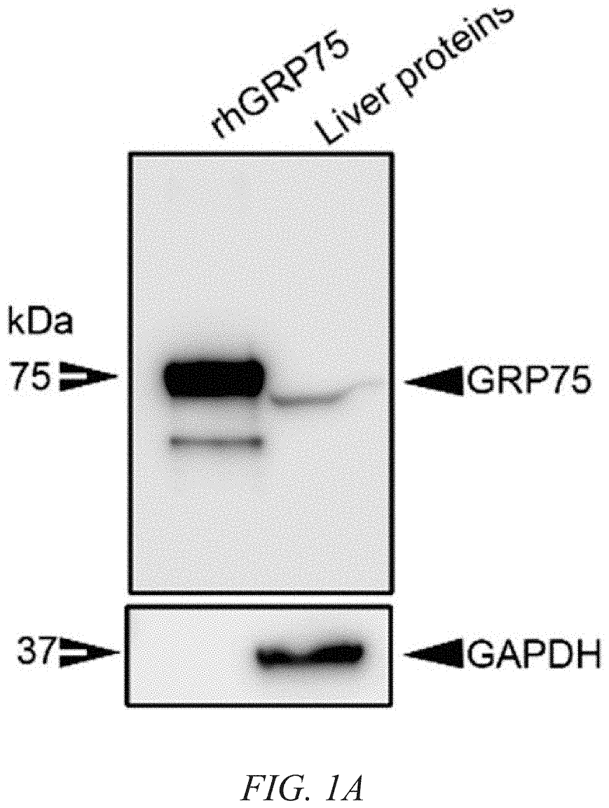

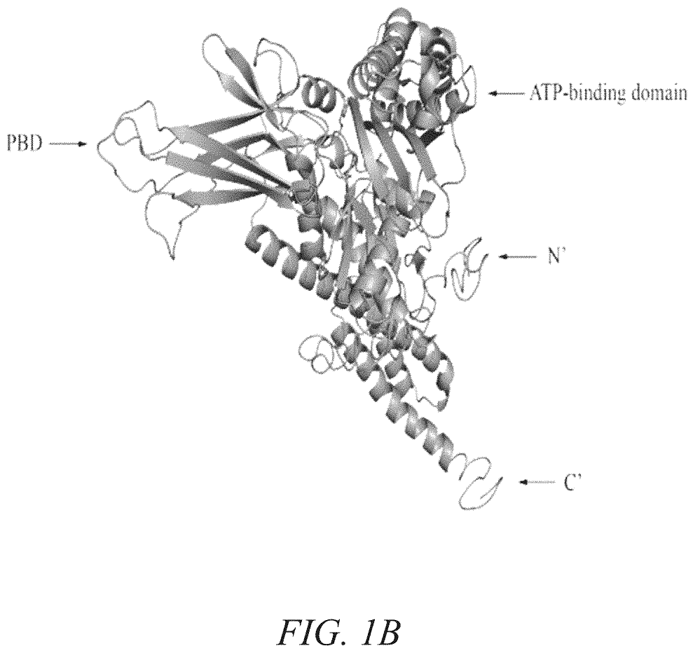

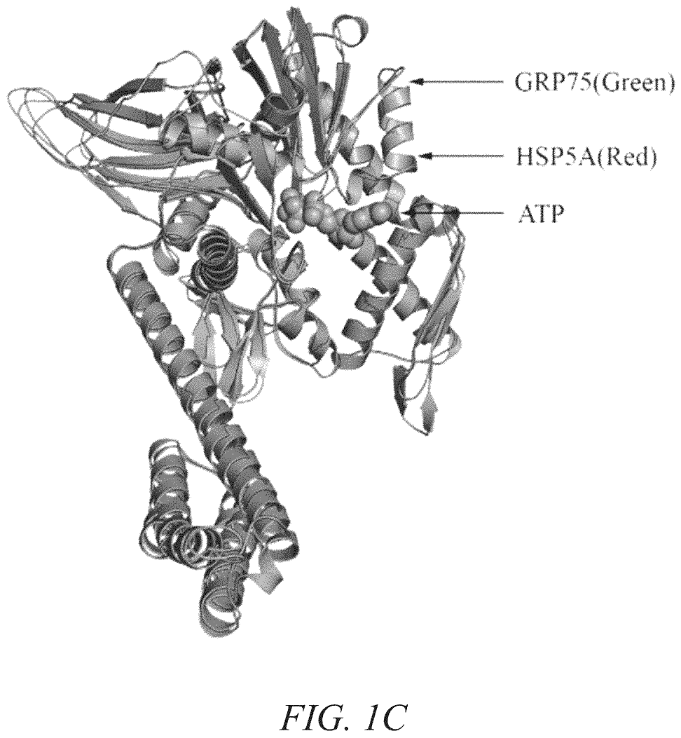

FIG. 1A illustrates, using a monoclonal anti-GRP75 antibody, immunoprecipitation method, and Western blot technique, precipitate of only one band at an expected size (75 kDa) as estimated by the molecular weight of standard marker and recombinant human GRP75 (rhGRP75, as a positive control). The liquid chromatography coupled with tandem mass spectrometry (LC-MS/MS) analysis showed, that chicken GRP75 consists of 675 amino acids, which were directly submitted to PRIDE and ProteomeXchange Knowledgebase (PRIDE, EMBL-EBI, Accession number # PXD013591, DOI 10.6019/PXDO13591). Three-dimensional structure prediction, through PyMOL homology modeling program, shows that chicken GRP75 contains ATP- and peptide-binding domains that covers position from amino acid residues 51 to 434 and 435-581, respectively (FIG. 1B). Besides the two principal domains (the N-terminal ATPase and C-terminal region) like most HSPs, chicken GRP75 structure match that of heat shock protein family A (HSPAS) with high homology (52.5%) (FIG. 1C) indicating that chicken GRP75 belongs to HSP70 family.

Sub-Cellular Localization of Chicken GRP75 Protein.

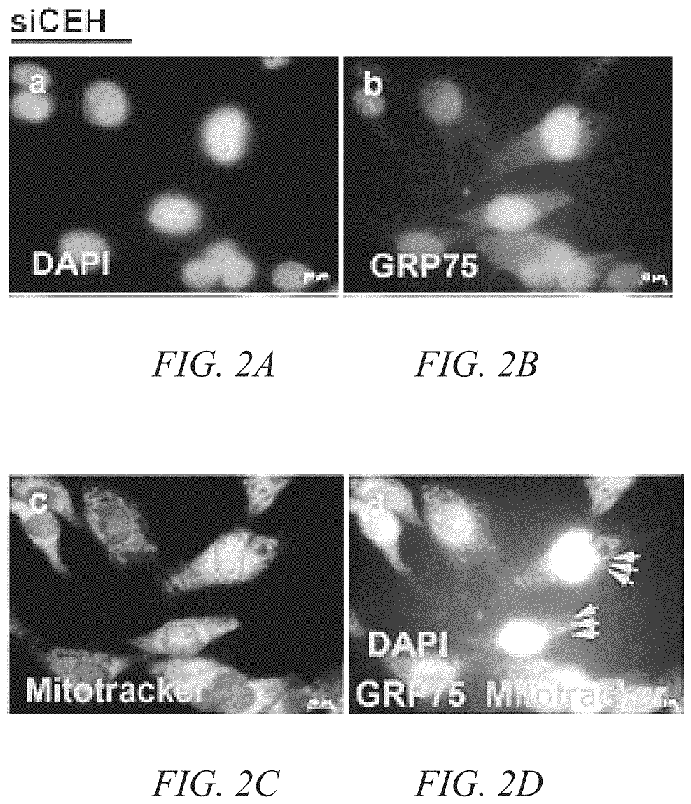

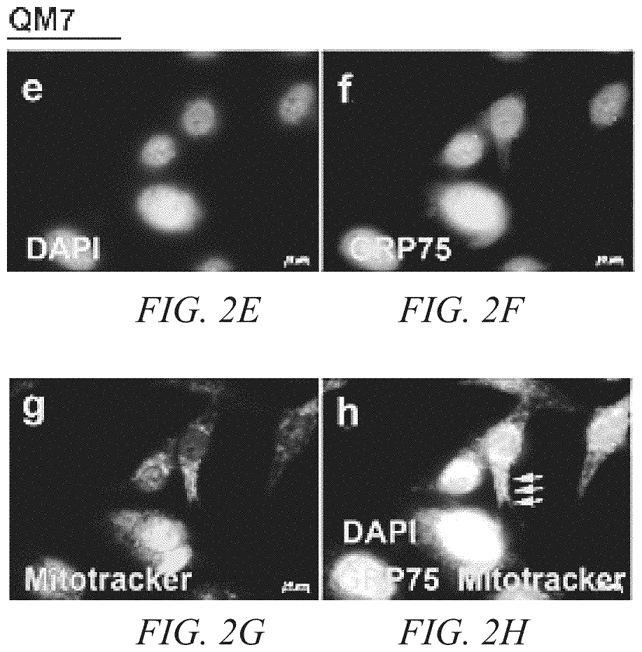

To gain further insights in GRP75 characterization and to determine its sub-cellular localization and distribution, double-labeling immunofluorescence was performed in two avian cell lines: spontaneously immortalized chicken embryonic hepatocyte (siCEH) and quail myoblast QM7. GRP75 was stained in combination with the well characterized mitochondrial fluorescent tracking dye, mitotracker, and shows to be predominantly localized in the mitochondria of hepatic and muscle avian cells (FIGS. 2A through 2H). As mitochondria is a primary site for ATP synthesis as well for reactive oxygen species production during heat stress exposure, GRP75 is a key regulator for heat stress response.

GRP75 is Responsive to Heat Stress.

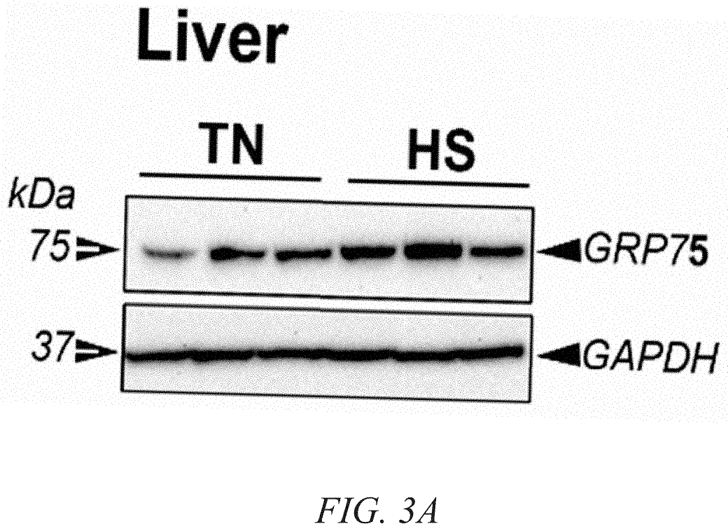

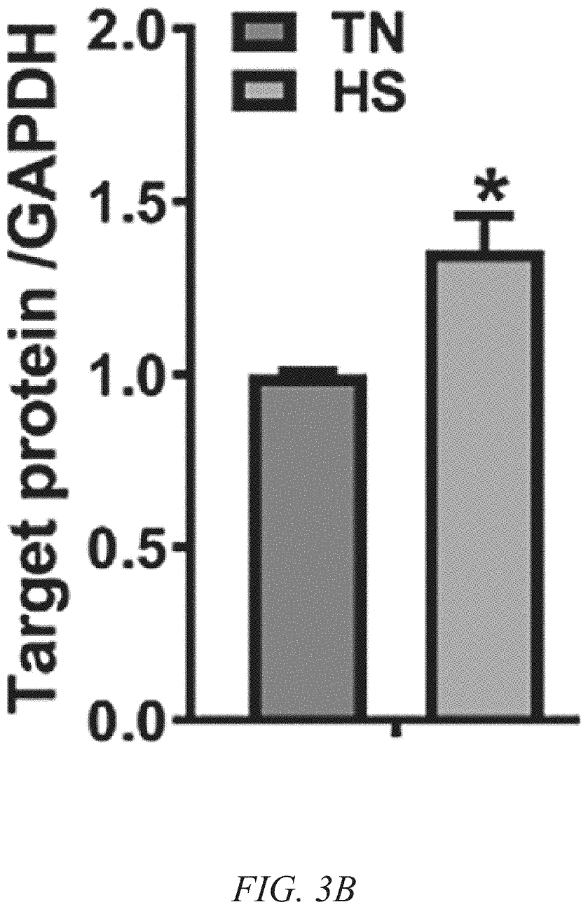

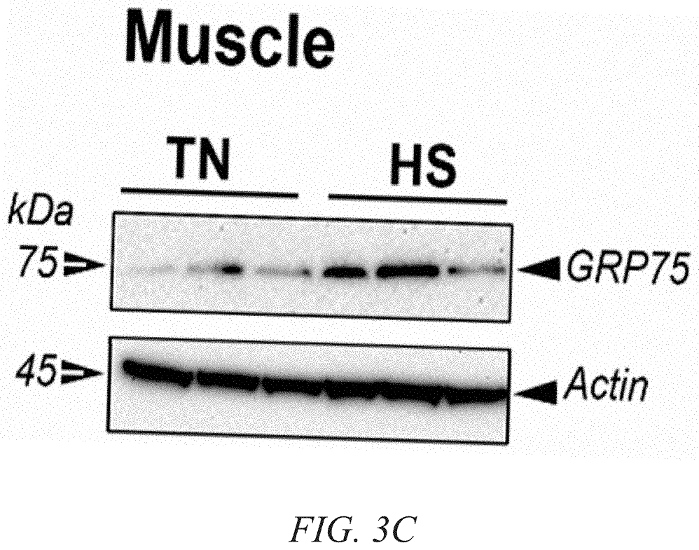

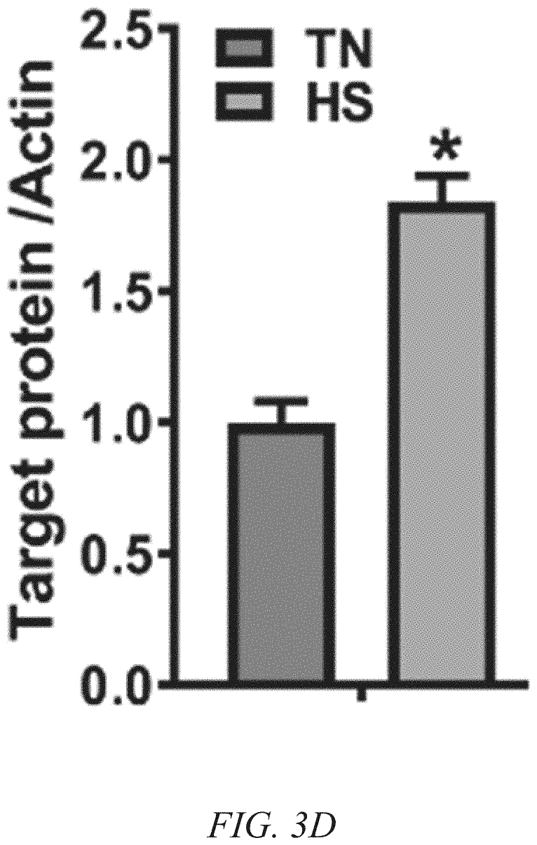

Broiler (meat-type) chickens were exposed to acute (2h) heat stress (35° C.) and GRP75 expression was analyzed in two metabolically important tissues (liver and muscle). Heat stress upregulates chicken GRP75 protein expression in liver (FIGS. 3A-3B) and muscle (FIGS. 3C-3D). Similarly, GRP75 expression was also induced in the hypothalamus, liver, and muscle of sensitive quail line exposed to acute (2 h, 35° C.) heat stress (data not shown). Although the hypothalamic expression seemed to be species-dependent, liver and muscle GRP75 is induced by acute heat stress in both avian species (chicken and quail). Liver is the major metabolic organ in the body, and it has a crucial role in carbohydrate, lipid, cholesterol, bile acids, and protein metabolism as well as in mineral and vitamin metabolism. The liver also plays a key role in antioxidant system and detoxification of drugs and xenobiotics. Muscle plays a pivotal role in thermogenesis and protein synthesis metabolism, to mention a few. The induction of hepatic and muscle GRP75 expression was also confirmed by using in vitro model (siCEH and QM7 cell lines). This suggests a direct effect of heat stress on GRP75 expression.

GRP75 has a Cell Survival/Death Function.

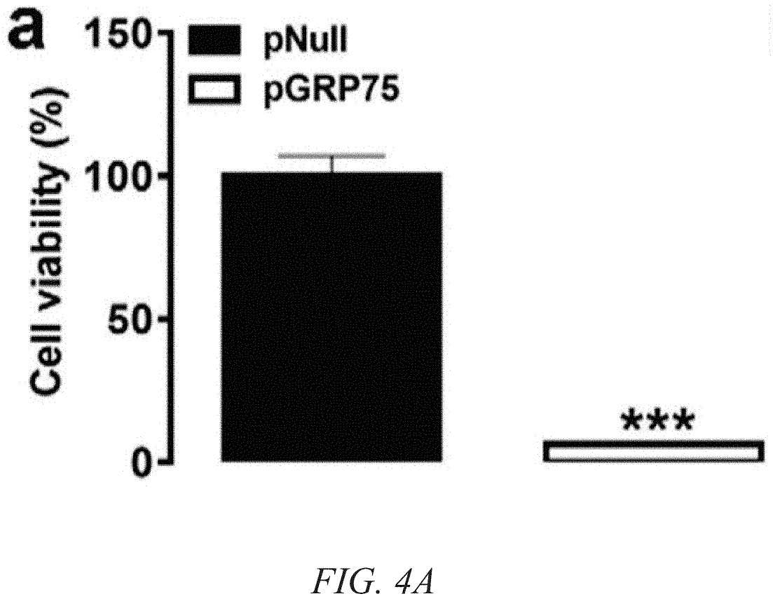

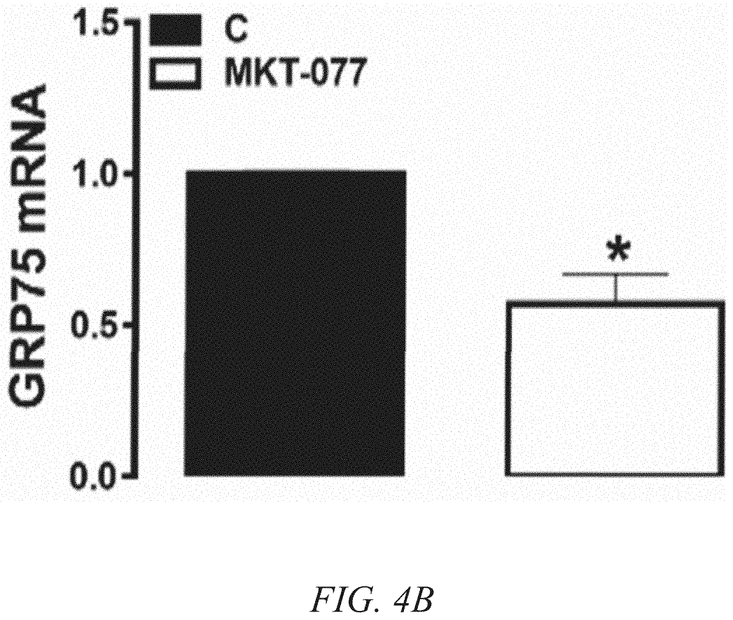

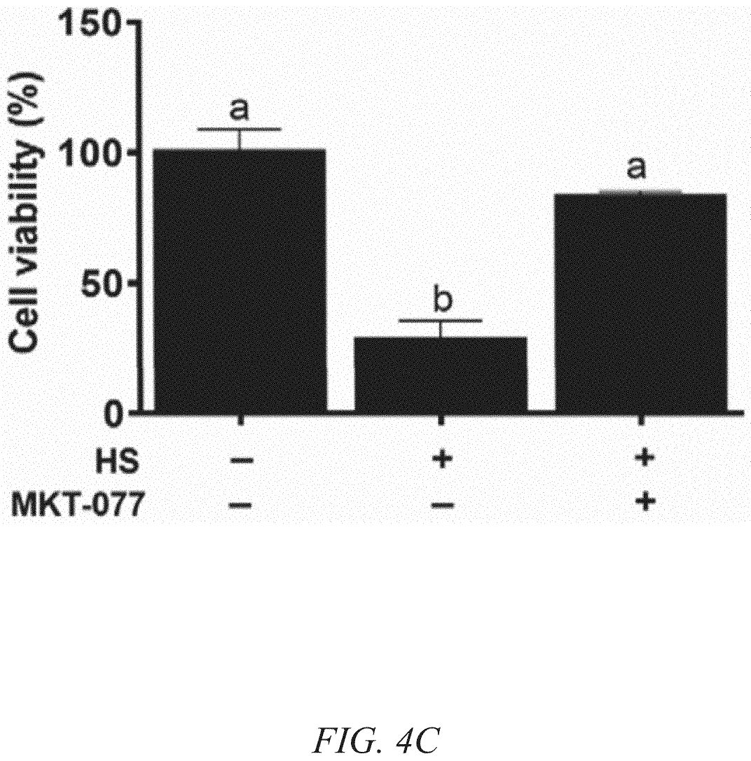

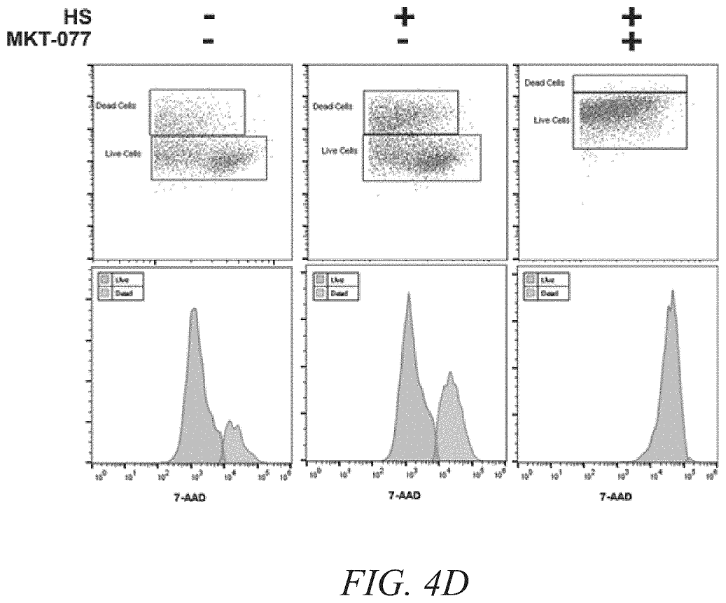

Depending on its duration, exposure, and severity, heat stress effects in poultry can range from discomfort (welfare), organ damage, to death by spiraling hyperthermia. The data showed that acute heat stress reduces siCEH and QM7 cell viability measured by 7-AAD staining and FACScan flow cytometer (FIGS. 4A-4D). GRP75 constitutes an essential component of the import machinery and protein quality control, regulates mitochondrial stress response signaling, and maintains mitochondrial homeodynamics. Overexpression of GRP75 alone reduces also siCEH and QM7 cell viability, and blockade of GRP75 by its small molecular inhibitor MKT-077 rescues the viability of both avian cell lines exposed to heat stress (FIG. 4A-4D).

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the instant disclosure may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Still further, additional aspects of the invention may be found in one or more appendices attached hereto and/or filed herewith, the disclosures of which are incorporated herein by reference as if fully set out at this point.

Thus, the invention is well adapted to carry out the objects and attain the ends and advantages mentioned above as well as those inherent therein. While the inventive concept has been described and illustrated herein by reference to certain illustrative embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A non-invasive method of detecting heat stress in an avian species, said method comprising the steps of:
(a) obtaining a sample from said avian species; and
(b) detecting the expression of GRP75 set forth in SEQ ID NO. 1 in said sample with an anti-GRP75 antibody and detecting binding between GRP75 and the antibody.

2. The method of claim 1 further comprising the step of assaying said sample for the expression level of a heat stress-related marker, said marker being a protein encoded by the gene of GRP75 or fragments of GRP75 which are immunoreacitve with an antibody that binds GRP75.

3. The method of claim 1 further comprising the step of determining the amount of GRP75 in the sample, wherein an elevated amount of GRP75 indicates the presence of heat stress in the avian species.

4. The method of claim 1 further comprising the step of administering a heat stress mitigation treatment to said avian species having expression of GRP75.

5. The method of claim 4 wherein the heat stress mitigation treatment comprises managerial treatment, nutritional treatment, or a combination of both.

6. The method of claim 5 wherein the managerial treatment comprises house design and location, restricted feeding, intermittent feeding, feeding during cool hours of the day, controlled environmental conditions, cooling systems, transporting, litter management, ventilation, providing cold water, or a combination thereof.

7. The method of claim 5 wherein the nutritional treatment comprises restricting feed, wet or dual feeding, adding fat in diets, increasing energy in rations, supplementing vitamins, minerals, and electrolytes, osmolytes, phytochemical, or a combination thereof.

8. The method of claim 4 further comprising the step of monitoring of heat stress response and well-being of said avian species over time during the heat stress mitigation treatment.

9. The method of claim 4 further comprising the step of measuring time-course, dynamic changes, or both of stress levels during the heat stress mitigation treatment.

10. The method of claim 1 wherein said avian species is poultry.

11. The method of claim 10 wherein said poultry is selected from the group consisting of chickens, turkeys, or quail.

12. The method of claim 11 wherein said poultry is a broiler chicken.

13. The method of claim 1 wherein said sample is non-invasively obtained without biopsy or euthanasia.

14. A polymerase chain reaction (PCR) kit for use with the method of claim 1.

15. An enzyme-linked immunosorbent assay (ELISA) kit for use with the method of claim 1.

16. A method of detecting and treating heat stress in a poultry species, said method comprising the steps of:
(a) non-invasively obtaining a sample from said poultry species;
(b) assaying said sample for the expression level of a heat stress-related marker, said marker being a protein encoded by the gene of GRP75 or fragments of GRP75 which are immunoreacitve to an antibody that binds GRP75, wherein expression of GRP75 is indicative of said heat stress in said poultry species; and
(c) administering a heat stress mitigation treatment to said poultry species having expression of GRP75.

17. The method of claim 16 wherein the heat stress mitigation treatment comprises managerial treatment, nutritional treatment, or a combination of both.

18. The method of claim 17 wherein the managerial treatment comprises house design and location, restricted feeding, intermittent feeding, feeding during cool hours of the day, controlled environmental conditions, cooling systems, transporting, litter management, ventilation, providing cold water, or a combination thereof.

19. The method of claim 17 wherein the nutritional treatment comprises restricting feed, wet or dual feeding, adding fat in diets, increasing energy in rations, supplementing vitamins, minerals, and electrolytes, osmolytes, phytochemical, or a combination thereof.

20. The method of claim 16 further comprising the step of monitoring of heat stress response and well-being of said poultry species over time during the heat stress mitigation treatment.

21. The method of claim 16 further comprising the step of measuring time-course, dynamic changes, or both of stress levels during the heat stress mitigation treatment.

22. The method of claim 16 wherein said poultry species is selected from the group consisting of chickens, turkeys, or quail.

23. A polymerase chain reaction (PCR) kit for use with the method of claim 16.

24. An enzyme-linked immunosorbent assay (ELISA) kit for use with the method of claim 16.


SEQUENCE LISTING (1 sequence)

SEQ ID NO: 1 - protein, 675 aa, Gallus gallus
MISASRAAARLPLLLPRGGPVPAVPGLAQTFWNGLSQNVLRAASSRKYASEAIKGAVIGIDLGTTNSCVAVMEGKQAKVLENSEGARTTPSVVAFTADGERLVGMPAKRQAVTNPHNTFYATKRLIGRRFDDSEVKKDIKNVPFKIVRASNGDAWVEAHGKLYSPSQIGAFVLMKMKETAENYLGHPAKNAVITVPAYFNDSQRQATKDAGQISGLNVLRVINEPTAAALAYGLDKSEDKIIAVYDLGGGTFDISILEIQKGVFEVKSTNGDTFLGGEDFDQALLQYIVKEFKRETSVDLTKDNMALQRVREASEKAKCELSSSVQTDINLPYLTMDASGPKHLNMKLSRSQFEGIVADLIKRTVAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMASDNKLLGQFTLVGIPPAPRGVPQIEVTFDIDANGIVHVSAKDKGTGREQQIVIQSSGGLSKDEIENMVKNAEKYAEEDRRRKERVEAVNLAEGIIHDTESKMEEFKDQLPADECNKLKEEIAKMRELLARKDTETGENIRQAATSLQQASLKLFEMAYKKMASERESSGSSGDQKEEKQ